(19) United States
(12) Patent Application Publication
(10) Pub. No.: US 2025/0378920 A1
(43) Pub. Date: Dec. 11, 2025

(54) SYSTEMS AND METHODS FOR DEVELOPING NOVEL MATERIALS

(71) Applicant: The Johns Hopkins University, Baltimore, MD (US)

(72) Inventor: Victor J. Leon, Silver Spring, MD (US)

(21) Appl. No.: 19/178,228

(22) Filed: Apr. 14, 2025

Related U.S. Application Data
(60) Provisional application No. 63/658,209, filed on Jun. 10, 2024.

(51) Int. Cl. G16C 60/00; G06F 30/27

(52) U.S. Cl. CPC G16C 60/00; G06F 30/27

(57) ABSTRACT

Described herein is a computer implemented method for generating novel material structures. The method incudes applying one or more transformations to a first material candidate to generate a second material candidate, where the one or more transformations are restricted by a set of constraints. Then, the first material candidate and second material candidate are scored using a scoring function. The method also includes truncating results of the scoring function based on a threshold value, which is related to uncertainty within the scoring function. Then, a best material candidate is chosen from the first material candidate or the second material candidate based on a score for each material candidate after the truncating. Finally, a material structure, which includes at least the best candidate material, is designed.

### CROSS-REFERENCE TO RELATED APPLICATIONS

The present application claims priority to and filing benefit of U.S. Provisional Patent Application No. 63/658,209, filed on Jun. 10, 2024, which is incorporated herein by reference in its entirety.

### BACKGROUND

Novel functional materials enable fundamental breakthroughs across technological applications from clean energy to information processing. Such materials are typically generated using computational models. However, current computational models for materials generation are limited due to their computational inefficiency and their inability to satisfy multiple objectives, satisfy hard constraints, and account for model uncertainty.

### SUMMARY

Provided herein are system, apparatus, device, method and/or computer program product aspects, and/or combinations and sub-combinations thereof for generating novel materials using a materials generation platform. The materials generation platform may be configured to generate materials with hard constraints and multiple simultaneous objectives while accounting for model uncertainty.

In some aspects, a computer implemented method may include applying one or more transformations to a first material candidate to generate a second material candidate. The one or more transformations may be restricted by a set of constraints. Then, the first material candidate and second material candidate are scored using a scoring function. The method also includes truncating results of the scoring function based on a threshold value, which is related to uncertainty within the scoring function. Then, a best material candidate is chosen from the first material candidate or the second material candidate based on a score for each material candidate after the truncating. Finally, a material structure, which includes at least the best candidate material, is designed.

### BRIEF DESCRIPTION OF THE DRAWINGS

The accompanying drawings are incorporated herein and form a part of the specification.

FIG. 1 shows an example material generation platform architecture, according to some aspects.

FIG. 2 shows a process for generating a novel material, according to some aspects.

FIG. 3 shows an example environment for generating a candidate material, according to some aspects.

FIG. 4 shows an example environment for scoring a candidate material, according to some aspects.

FIGS. 5A and 5B show an example plot for an example environment for applying uncertainty to a score of one or more candidate materials, according to some aspects.

FIG. 6 shows a process for implementing active learning in a materials generation platform, according to some aspects.

FIG. 7 shows a computer system, according to some aspects.

In the drawings, like reference numbers generally indicate identical or similar elements. Additionally, generally, the left-most digit(s) of a reference number identifies the drawing in which the reference number first appears.

### DETAILED DESCRIPTION

The aspects described herein, and references in the specification to “one aspect,” “an aspect,” “an exemplary aspect,” “an example aspect,” etc., indicate that the aspects described can include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of those skilled in the art to effect such feature, structure, or characteristic in connection with other aspects whether or not explicitly described.

The terms “about,” “approximately,” or the like can be used herein indicates the value of a given quantity that can vary based on a particular technology. Based on the particular technology, the terms “about,” “approximately,” or the like can indicate a value of a given quantity that varies within, for example, 10-30% of the value (e.g., +10%, +20%, or +30% of the value).

Aspects of the present disclosure can be implemented in hardware, firmware, software, or any combination thereof. Aspects of the disclosure can also be implemented as instructions stored on a computer-readable medium, which can be read and executed by one or more processors. A machine-readable medium can include any mechanism for storing or transmitting information in a form readable by a machine (e.g., a computing device). For example, a machine-readable medium can include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Furthermore, firmware, software, routines, and/or instructions can be described herein as performing certain actions. However, it should be appreciated that such descriptions are merely for convenience and that such actions result from computing devices, processors, controllers, or other devices executing the firmware, software, routines, instructions, etc. The term “machine-readable medium” can be interchangeable with similar terms, for example, “computer program product,” “computer-readable medium,” “non-transitory computer-readable medium,” or the like. The term “non-transitory” can be used herein to characterize one or more forms of computer readable media except for a transitory, propagating signal.

Provided herein are system, apparatus, device, method and/or computer program product aspects, and/or combinations and sub-combinations thereof for generating novel materials using a materials generation platform. The materials generation platform may be configured to generate materials with hard constraints and multiple simultaneous objectives while accounting for model uncertainty.

The design of functional materials with desired properties is essential in driving technological advances. Computational models are commonly used to generate and screen such materials. In one example, high-throughput density functional theory (DFT) screenings may filter a pool of candidate materials based on a set of desired properties. In another example, machine learning models, such as graph neural networks, predict properties (e.g., stability) of a generated candidate material. The true properties of the candidate material are then calculated using DFT and used to tune the machine learning model. In another example, a machine learning based diffusion model generates candidate materials by gradually refining atoms, coordinates, and a periodic lattice of an initial structure through a slow diffusive de-noising process.

A primary technical challenge for current computational models is simulating materials with greater than thirty atoms per unit cell (i.e., the smallest repeating structural unit that, when replicated in all directions, can build an entire crystal structure of a material). DFT based calculations are typically limited to materials with about thirty atoms per unit cell due to computational costs, while generative models may become unstable when simulating atoms with greater than 20 atoms per unit cell. This limits the classes of materials that may be explored by these models. For example, current models struggle to simulate spinels, pyrochlores, and garnets.

Furthermore, current computational models may suffer from technical limitations related to applying constraints during simulations. For example, machine learning models may be unable to impose hard constraints, such as structural symmetry, during a materials generation simulation. As a result, these models may disproportionately generate non-symmetric material structures.

Current computational models may also suffer from technical limitations in related to integrating uncertainty. For example, current DFT and machine learning based models may not account for uncertainty due to differences between computational and experimental values. This may lead to simulations that explore a smaller materials space, and thus generate fewer novel results.

Embodiments disclosed herein solve common technical problems associated with computational models for materials generation.

In an aspect, the materials generation platform described herein applies one or more transformations to a first material candidate to generate a second material candidate. The one or more transformations may be restricted by a set of constraints. Then, the materials generation platform scores the first material candidate and the second material candidate using one or more scoring models. To account for uncertainty in the scoring models, the materials generation platform truncates results of the scoring function based on a threshold value. Then, the materials generation platform chooses a best material candidate from the first material candidate and the second material candidate based on the score of each material candidate after the truncating. Finally, the materials generation platform designs a material structure, which includes at least the best candidate material.

In an aspect, these approaches provide direct technological improvements over previous systems via an implementation that simultaneously satisfies hard constraints, considers multiple objectives, and accounts for uncertainty. In an aspect, the approaches described herein also improve functioning of a computer system. For example, the use of scoring models to evaluate a candidate material across multiple objectives can save computational time and resources that would have been expended to perform computationally expensive DFT calculations. The conservation of computational time and resources allows for the exploration of a larger number of candidate materials. Furthermore, while the conservation of computational and memory resources may be limited with respect to a single client device, the total conservation of computational and memory resources across an entire fleet of client devices may be significant. In one aspect, these technical improvements may be appreciated, for example, in resource-constrained environments. In an aspect, the overall computational efficiency of these systems may be improved as a result and the conserved resources may be reallocated for other tasks. Additionally, the implementation of uncertainty within a materials generation model may lead to fewer computational errors and higher performance accuracy.

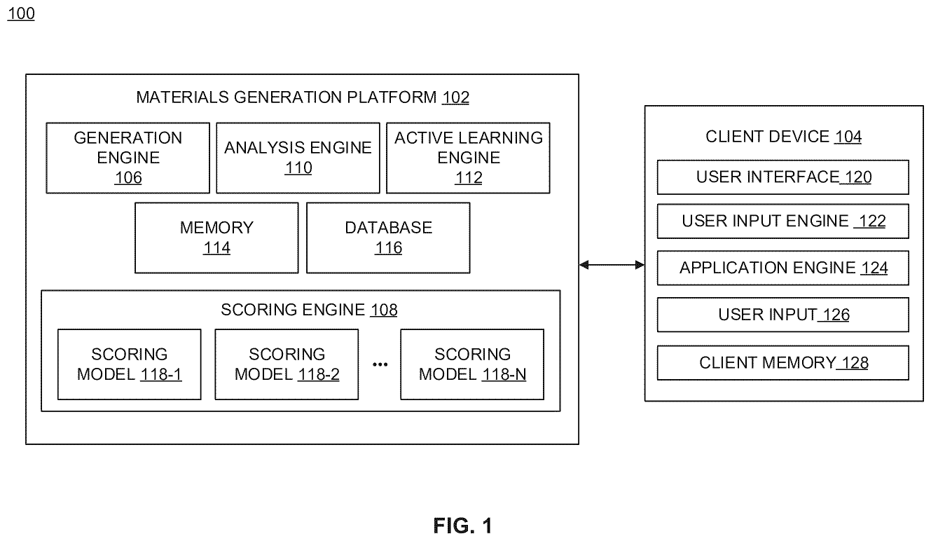

Various aspects of this disclosure may be implemented using and/or may be a part of the example materials generation platform shown in FIG. 1. It is noted, however, that these environments are provided solely for illustrative purposes, and are not limiting. Aspects of this disclosure may be implemented using and/or may be part of environments different from and/or in addition to the materials generation platform, as will be appreciated by persons skilled in the relevant art(s) based on the teachings contained herein.

An example of the materials generation platform shall now be described.

FIG. 1 shows a block diagram of an example materials generation platform architecture 100, according to some aspects. Operations described may be implemented by processing logic that may comprise hardware (e.g. circuitry, dedicated logic, programmable logic, microcode, etc.), software (e.g. instructions executing on a processing device), or a combination thereof. It is to be appreciated that not all operations may be needed to perform the disclosure provided herein. Further, some of the operations may be performed simultaneously, or in a different order than described for FIG. 1, as will be understood by a person of ordinary skill in the art.

Example materials generation platform architecture 100 may include a material generation platform 102 and a client device 104. In some aspects, example material generation platform architecture 100 may be implemented partially or entirely at client device 104. Alternatively or additionally, in some aspects, example materials generation platform architecture 100 may be implemented partially or entirely at third party servers or within the cloud. In such aspects, client device 104 and materials generation platform 102 may be communicatively coupled with each other via one or more networks, such as one or more wired or wireless local area networks (“LANs,” including Wi-Fi, mesh networks, Bluetooth, near-field communication, etc.) or wide area networks (“WANs”, including the Internet).

In some aspects, materials generation platform 102 may include a generation engine 106, a scoring engine 108, an analysis engine 110, an active learning engine 112, a memory 114, and a database 116. In some aspects, materials generation platform 102 may be implemented as one or more servers and/or one or more cloud servers. Materials generation platform 102 may also be implemented as a variety of centralized or decentralized computing devices. For example, materials generation platform 102 may operate on a mobile device, a laptop computer, a desktop computer, grid-computing resources, a virtualized computing resource, cloud computing resources, peer-to-peer distributed computing devices, a server farm, or a combination thereof. Materials generation platform 102 may be centralized in a single device, distributed across multiple devices within a cloud network, distributed across different geographic locations, or embedded within a network.

In some aspects, generation engine 106 may apply one or more transformations to a candidate material. As described herein, “candidate material” may refer to a unit cell of a material under consideration. A “unit cell” is the smallest repeating structural unit that, when replicated in all directions, can build an entire crystal structure of a material. The one or more transformations may include elementary operations that are performed on the unit cell to generate a new structure. Examples of elementary operations may include swapping an atom for another element, adding atoms to a unit cell, removing atoms from a unit cell, changing from one structure to another structure (e.g., from a perovskite to a pyrochlore), and the like. In some aspects, generation engine 106 may limit the one or transformations based on a set of constraints. The set of constraints may, as non-limiting examples, limit atomic swaps to a subset of chemical elements or limit the unit cell to certain structures, such as cubic structures or symmetric structures.

In some aspects, scoring engine 108 may leverage one or more scoring models 118-1 to 118-N (N being any positive, whole integer greater than 1; collectively scoring models 118) to score candidate materials. Each scoring model 118 may calculate a desired parameter of a candidate material. For example, scoring model 118-1 may calculate the stability, scoring model 118-2 may calculate band gap, scoring model 118-3 may calculate transparency, etc. Scoring models 118 may include fast and/or computationally inexpensive simulations with known or estimated uncertainties, machine learning and AI models, correlations, and others. In some aspects, scoring engine 108 may combine output values from each of the scoring models 118 into a combined score. For example, scoring engine 108 may perform any combination of normalization, addition, subtraction, multiplication, and division on the output values of scoring models 118-1 to 118-N. Scoring engine 108 may leverage any combination of one or more of scoring modes 118-1 to 118-N to score a candidate material. The combination of scoring models may depend on the desired properties of a candidate material and some scoring models may not be used.

In some aspects, analysis engine 110 may perform multiple functions within materials generation platform 102. In some aspects, analysis engine 110 applies uncertainties to combined scores generated by scoring engine 108. For example, analysis engine 110 may determine a threshold value that accounts for uncertainty within scoring models 118-1 to 118-N. Analysis engine 110 may then truncate any value that falls above (or below) the threshold value, depending on whether the threshold value is a maximum or minimum. Analysis engine 110 may also compare scores for at least two candidate materials to determine a best material. When a high score is favorable, the best material may be the material with the highest score. Alternatively, when a low score is favorable, the best material may be the material with the lowest score.

In some aspects, active learning engine 112 may update probabilities associated with the one or more transformations. For example, after generation engine 106 applies a transformation to a first candidate material to generate a second candidate material, active learning engine 112 may compare the scores of the first and second candidate materials. If the transformation results in a better score, active learning engine 112 may increase a probability of applying the transformation. Alternatively, if the transformation results in a worse score, active learning engine 112 may reduce the probability of applying the transformation.

In some aspects, active learning engine 112 may compare the score of the second candidate material to a threshold. The threshold may be calculated from a subset of scoring models 118-1 to 118-N. For example, the threshold may be calculated from scoring models 118-1, 118-2, and 118-5 instead of the full set of scoring models 118-1 to 118-N. Active learning engine 112 may increase or decrease a probability of applying a transformation based on whether the score of the second candidate material is above or below the threshold. In one non-limiting example, the second candidate material may be generated by applying a set of transformations to the first candidate material. Then, a combination of one or more of scoring models 118 may estimate the stability of the second candidate material. If the score is above a threshold, the second candidate material is less likely to be stable, and active learning engine 112 may decrease a probability of applying the set of transformations. If the score is below the threshold, the second candidate material is more likely to be stable and active learning engine 112 may increase a probability of applying the set of transformations.

In some aspects, memory 114 may store variables and operations during a materials generation process. For example, memory 114 may store previous transformations that have been applied to candidate materials, thus ensuring that calculations are not repeated. Memory 114 may also store a probability database that gives a transformations probability of success.

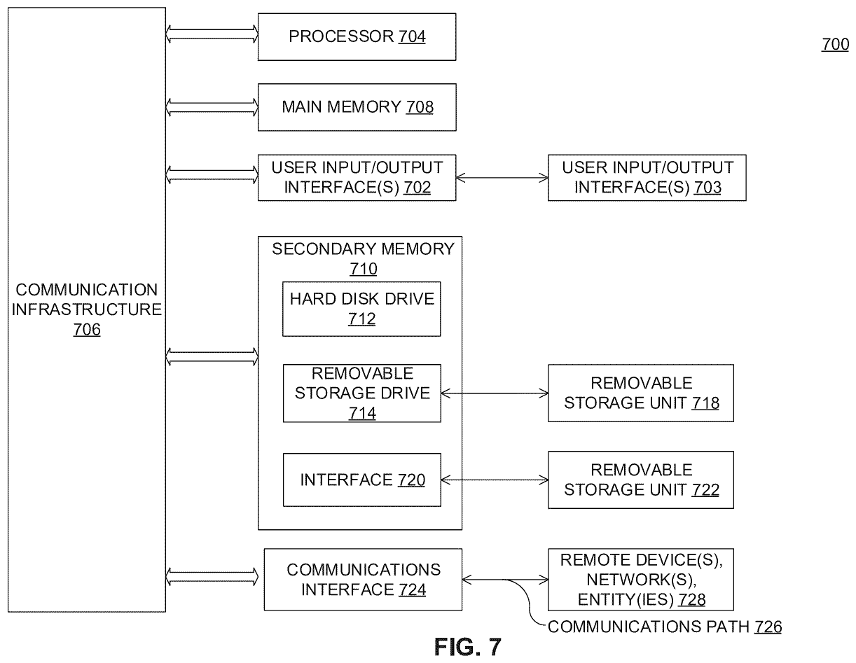

In some aspects, database 116 may store various data used and/or generated by materials generation platform 102, including transformations, constraints, and generated materials. Database 116 may be stored, for example, in a volatile memory (e.g. random access memory (RAM)), a non-volatile storage device (e.g. a disk), or in a distributed and/or redundant manner across multiple memories and/or storage devices. In some aspects, database 116 is managed by and accessed via a corresponding database management system (DBMS), which is not shown in FIG. 1 for the sake of simplicity. Database 116 and the corresponding DBMS may be implemented on one or more computer systems, such as computer system 700 as described below in reference to FIG. 7. Database 116 and the corresponding DBMS may also be implemented on one or more servers of an enterprise network and/or a cloud computing network.

In some aspect, client device 104 may be one or more of a desktop computer, a laptop computer, a tablet, or a mobile phone. Additional and/or alternative client devices may be contemplated. Client device 104 may include a corresponding user interface 120, user input engine 122, application engine 124, user input 126, and client memory 128.

In some aspects, user interface 120 may be configured to render content including unimodal responses, multimodal responses, or other content for audible or visual presentation to a user of client device 104 using one or more user interface output devices. For example, client device 104 may include a display or projector that enables content to be provided for visual presentation to a user via client device 104. Alternatively or additionally, client device 104 may include one or more speakers that enable content to be provided for audible presentation to a user via client device 104.

In some aspects, application engine 124 may execute one or more software applications on client device 104. In some aspects, application engine 124 may submit a user input (e.g., user input 126) to material generation platform 102. Application engine 124 may then receive unimodal, multimodal, or other responses from materials generation platform 102 in response to a natural language query, which may then be rendered onto user interface 120 (e.g., audibly and/or visually). Application engine 124 may execute one or more software applications that are separate from an operating system of the client device 104 or may alternatively be implemented directly by the operating system of client device 104. For example, the application engine 124 may execute one or more software applications via a web browser or assistant.

In some aspects, user input 126 may represent an input provided by a user of client device 104 and may be detected via user input engine 122. For example, user input 126 may include initial parameters for conducting a material generation simulation, such as constraints and a starting candidate material. In some aspects, user input 126 may be a typed query that is typed via a physical or virtual keyboard, a suggested query that is selected via a touch screen or a mouse of client device 104, a spoken voice query that is detected via a microphone of client device 104 (or directed to a voice assistant running at client device 104), or an image or video query that is based on vision data captured by a vision component of client device 104.

In some aspects, client memory 128 may include a data store containing data about a user of client device 104 or about client device 104 itself. In some aspects, client memory 128 may store one or more user inputs (e.g., user inputs 126) made by a user of client device 104. Client memory 128 may also store a context of client device 104. Client memory 128 may also store user interaction data about current or recent interactions between a user or multiple users and client device 104. In some aspects, client memory 128 may also store location data about current or recent locations of client device 104 or a geographical region associated with a user of client device 104. Client memory 128 may also store user attribute data, user preference data, a user profile, or various configurations relating to client device 104 or a user of client device 104. In some aspects, the data stored in client memory 128 may be communicated partially or entirely to materials generation platform 102 (e.g. to produce higher quality outputs).

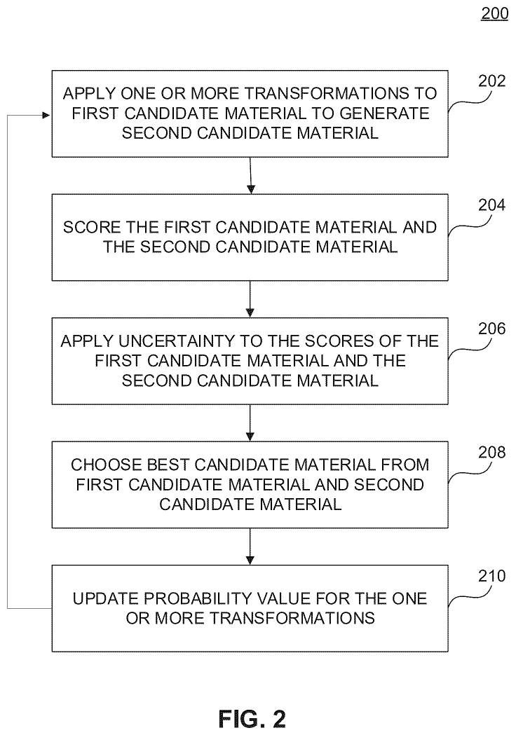

FIG. 2 shows a flowchart of a process 200, according to some aspects. Process 200 may, for example, describe a process for generating novel materials. Operations described may be implemented by processing logic that may comprise hardware (e.g., circuitry, dedicated logic, programmable logic, microcode, etc.), software (e.g., instructions executing on a processing device), firmware or a combination thereof. It is to be appreciated that not all operations may be needed to perform the disclosure provided herein. Further, some of the operations may be performed simultaneously, or in a different order than described for FIG. 2, as will be understood by a person of ordinary skill in the art. Process 200 shall be described with reference to FIG. 1. However, process 200 is not limited to that example aspect.

At 202, a materials generation platform (e.g., materials generation platform 102) may apply one or more transformations to a first candidate material to generate a second candidate material. For example, the materials generation platform may leverage a generation engine (e.g., generation engine 106) to apply one or more elementary transformations to the first candidate material. Elementary transformations may include, but are not limited to, swapping atoms, adding atoms to a structure, removing atoms from a structure, changing from one structure to another structure (e.g., from a perovskite to a pyrochlore), etc. In an initial iteration of process 200, the first candidate material is a starting material. The starting material may contain desirable properties and/or may be easily transformed to generate other material structures. In subsequent iterations of process 200, the first material may be a best material determined during a prior process 200 (see step 208 below).

At 204, the materials generation platform may score the first candidate material and the second candidate material. For example, the materials generation platform may leverage a scoring engine (e.g., scoring engine 108) to score each candidate material using one or more scoring models (e.g., scoring models 116). The scoring models may each computationally determine a parameter of interest. For example, when generating electrically insulative materials, parameters of interest may include stability and band gap. The scoring engine may also estimate uncertainty for each of the scoring models. In some aspects, the uncertainty is estimated using statistical parameters, such as root mean square error (RMSE) and the like. Additionally or alternatively, the uncertainty for a scoring model may be known. For example, a scoring model may consistently overestimate band gap of a material by about 10%.

At 206, the materials generation platform may apply uncertainty to the scores of the first candidate material and the second candidate material. For example, an analysis engine (e.g., analysis engine 110) may check the scores against a threshold value. If the threshold value is a minimum, the analysis engine may raise any scores below the threshold value. Similarly, if the threshold value is a maximum, the analysis engine may lower any scores above the threshold value. For example, if a score for the first candidate material is 3.2 and the threshold value is 2.2, the score for the first candidate material is lowered to 2.2. In some aspects, the threshold value may be derived from the estimated uncertainty of the scoring models using, for example, error propagation techniques.

In some aspects, the threshold value is applied to the difference between scores of the first candidate material and the second candidate material. A larger magnitude in difference between the first candidate material's score and the second candidate material's score may indicate that there is a larger difference in performance between the materials. Thus, a threshold of minimum magnitude difference for a significant difference can be applied. This threshold may be calculated for a predictive model (e.g., a scoring model) or from a subset of scoring models 118-1 to 118-N. The predictive model may predict a property of interest, such as band gap. Then, the output of the predictive model may be compared to a ground truth value, such as a DFT calculation, an experimental value, or a test statistic. For example, mean squared error (MSE) may be calculated between output of the predictive model and the ground truth value and used as the threshold value for the difference between scores. In one non-limiting example, a scoring function for estimating band gap may have a measured MSE of 1 eV, so 1 eV is used as a threshold for the comparison between the first and second material. The predicted scores for the first material and second material are 2 and 1.5 eV. The difference is 0.5 eV, which is below the threshold, meaning the difference between the two materials is insignificant.

At 208, the platform may choose a best candidate material from the first candidate material and the second candidate material. In some aspects, the best candidate material may be the material with the highest (or lowest) score after uncertainty is applied. For example, in a scenario where a higher score is desirable, the first candidate material has a score of 1.8, and the second candidate material has a score of 2.2, the analysis engine may choose the second candidate material as the best candidate material. In another example, when the first and second candidate materials have identical scores, the analysis engine may randomly choose one of the first candidate material and second candidate material as the best candidate material.

In another example, when the threshold is applied to a difference in scores between the first candidate material and the second candidate material and the difference is greater than the threshold, the best candidate material may be determined from the sign (e.g., positive or negative) of the difference between the scores of the first and second candidate materials. For example, if a lower score is desirable, and a difference calculated by subtracting the score of the first candidate material from the score of the second candidate material is negative, the second candidate material is chosen as the best material. Alternatively, if the difference calculated by subtracting the score of the first candidate material from the score of the second candidate material is positive, the first candidate material is chosen as the best material. In some aspects, when the magnitude of the difference is insignificant (i.e., less than or equal to the threshold), the analysis engine may randomly choose one of the first or second candidate materials as the best material or the analysis engine may default to either the first or second candidate material as the best material.

At 210, the materials generation platform updates a probability value for the transformations applied to the first candidate material at 202. For example, if the one or more transformations resulted in the second material having a better score than the first material, the probability value may increase. Alternatively, if the one or more transformations results in the second materials having a worse score than the first material, the probability value may decrease. In some aspects, when the scores of the first material and the second material are identical the probability value may not change. In some aspects, the materials generation platform may also update the threshold value. For example, when the goal of the materials generation platform is to minimize the score, the threshold value may decrease via a schedule during iterations of process 200. In this case, the threshold value is a minimum, and the threshold may decrease according to:

$${τ}_{i} = {τ}_{0} ( 1 - \frac{i}{n} ) ,$$

where τ₀ is an initial threshold, i is the current iteration of process 200, and n is the total number of iterations of process 200 that will be performed during a materials generation process. When τᵢ is large (e.g., at the beginning of a set of processes), the materials generation platform may explore a broader materials space. Then as τᵢ decreases (e.g., towards the middle and end of a set of processes), the materials generation platform may concentrate on a narrower range of “good” material candidates. A user of the materials generation platform may tune τ₀ and n to balance exploration vs. exploitation of a search space (e.g., material candidates).

In some aspects, process 200 is repeated several times. For example, process 200 may be repeated for a set number of iterations or for a set amount of time. In each successive iteration of process 200, the first candidate material is replaced with the best candidate material determined at a previous step 208. Multiple iterations of process 200 may be implemented in parallel.

While process 200 is described herein in the context of materials generation, it will be understood by a person of ordinary skill in the art that process 200 may be implemented for any engineering design problem wherein a user can define elementary operations for generating new candidates and define one or more scoring models that are computationally efficient and have a known (or estimated) degree of uncertainty.

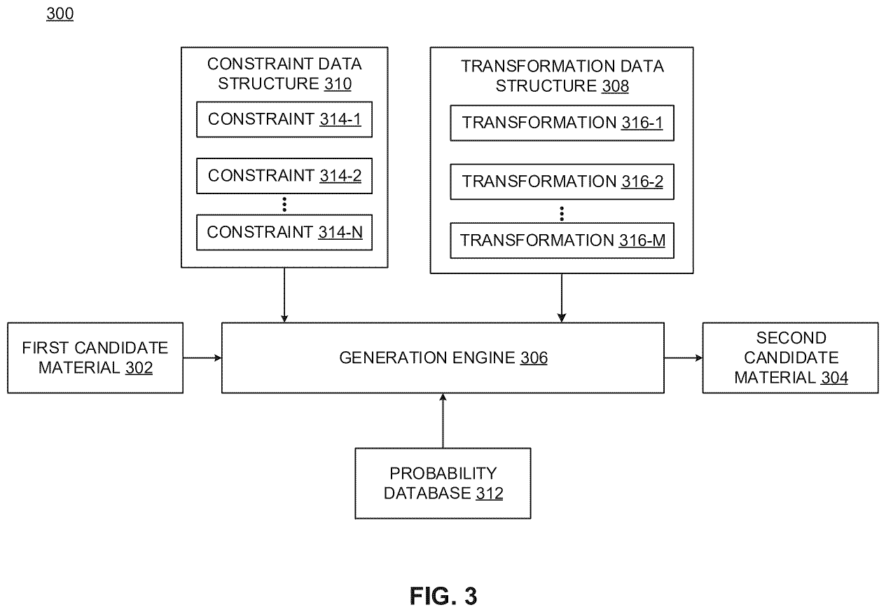

FIG. 3 illustrates an environment 300, according to some aspects. Environment 300 may describe a method for generating a second candidate material from a first candidate material. Operations described may be implemented by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously, or in a different order than described for FIG. 3, as will be understood by a person of ordinary skill in the art. Environment 300 shall be described with reference to FIGS. 1 and 2. However, environment 300 is not limited to these example aspects.

In some aspects, environment 300 may include a first candidate material 302, a second candidate material 304, a generation engine 306, a transformation data structure 308, a constraint data structure 310, and a probability database 312. Transformation data structure 310 may include transformations 314-1 to 314-N (N being a positive whole integer greater than 1; collectively transformations 314). Transformations 314 may include elementary operations that may be performed on first candidate material 302 to generate second candidate material 304. For example, transformations 314 may include swapping atoms, adding atoms, removing atoms, and the like. Constraint data structure 310 may include constraints 316-1-316-M (M being a positive whole integer greater than 1; collectively constraints 316). Constraints 316 may limit transformations 314. For example, constraints 316 may limit atom swapping to a subset of atomic elements (e.g. to a subset of elements with matching oxidation states). Constraints 316 may also limit the types of structures (e.g., symmetric structures, cubic structures) or class of materials (e.g., perovskites, spinels, pyrochlores, garnets, etc.) that are produced by generation engine 306. Additionally, constraints 316 may limit candidate materials produced by generation engine 306 to materials with certain properties, such as conductivity, temperature resistance, chemical resistance, etc.

In some aspects, environment 300 receives first candidate material 302. First candidate material 302 may be a starting material, or the best material chosen in during a previous iteration of process 200 (e.g., 208 in FIG. 2). First candidate material 302 may be input into generation engine 306. Generation engine 306 may then fetch one or more transformations 314 from transformation data structure 308. The one or more transformations 314 may be fetched at random. In some aspects, each transformation 314 is weighted by a probability, such that transformations with a higher probability are more likely to be fetched and applied to first candidate material 302. In some aspects, transformation probabilities may depend on factors such as the element being swapped. For example, consider the material Mg₃Al₂(SiO₄)₃. Swapping Mg for Fe may have a different probability than swapping Al for Fe. In another example, swapping Mg for Fe in a perovskite structure may have a different probability than swapping Mg for Fe in a pyrochlore structure. Probabilities for each transformation may be stored in probability database 312. Generation engine 306 may apply the one or more of transformations to first candidate material 302, within the bounds of constraints 316, to generate second candidate material 304.

In the examples above, transformations and constraints for generating new candidate materials have been described. However, these examples are not meant to be limiting nor meant to represent an exhaustive list of possible implementations. Specifically, the concepts described herein may be applied to other engineering design problems where a user can conceive of simple operations to generate new samples. For example, if materials generation platform 102 were applied to the design of an airplane wing, one or more transformations may include manipulating the geometry and placement of the airplane wing. Constraints may include the width and location of the airplane wing. The scope of the technology disclosed herein is not limited to only these examples, and other implementations are contemplated as appreciated by one skilled in the art.

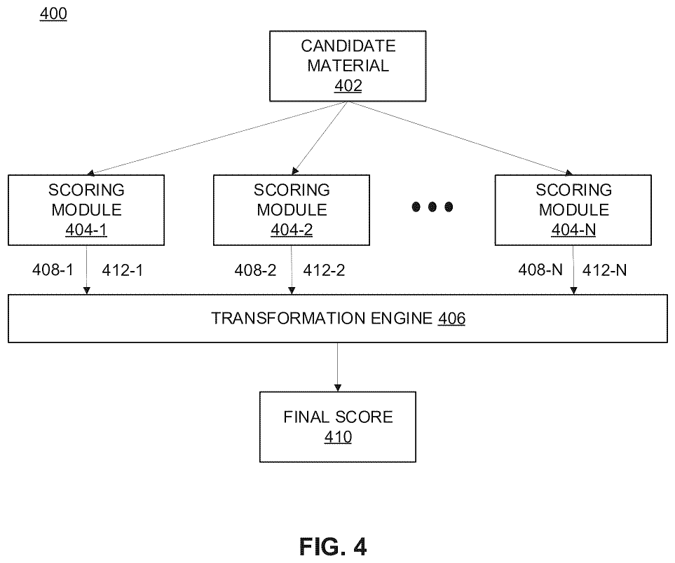

FIG. 4 illustrates an environment 400, according to some aspects. Environment 400 may describe a method for scoring a candidate material. Operations described may be implemented by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously, or in a different order than described for FIG. 4, as will be understood by a person of ordinary skill in the art. Environment 400 shall be described with reference to FIGS. 1-3. However, environment 400 is not limited to these example aspects.

Environment 400 may include a material candidate 402, scoring models 404-1 to 404-N (N being a positive whole integer greater than 1; collectively scoring models 404), and a transformation engine 406. Scoring models 404-1 to 404-N and transformation engine 406 may collectively form a scoring engine (e.g., scoring engine 108).

In some aspects, environment 400 may receive candidate material 402. Candidate material 402 may be an example of first candidate material 302 and/or second candidate material 304 of FIG. 3. During scoring, candidate material 402 is input into scoring models 404. Scoring models 404 may each calculate a different property of candidate material 402. For example, scoring model 404-1 may calculate stability of candidate material 402, while scoring model 404-2 calculates the band gap of candidate material 402. In some aspects, scoring models 404 may include computationally inexpensive simulations with a known or estimated amount of uncertainty. In some aspects, the uncertainty in a scoring model is unknown. Furthermore, scoring models 404 may be chosen to steer the materials generation process towards one or more objectives. For example, when generating electrically insulative materials, objectives may include stability and a target electrical resistance and scoring models 404 may include a stability calculation model 404-1 and a band gap calculation model 404-2 (resistance is generally related to the band gap of a material). In another example, when generating materials for high temperatures, objectives may include stability, electrical resistance, and temperature resistance and scoring models 404 may include a stability calculation model 404-1, an electrical resistance model 404-2, and a temperature resistance model 404C.

In environment 400, each scoring model 404-1 to 404-N may output a score 408-1 to 408-N (N being a positive whole integer greater than 1; collectively scores 408). Scores 408 are input into transformation engine 406. Transformation engine 406 may perform one or more operations on scores 408. For example, transformation engine 406 may first normalize scores 408. Then transformation engine 406 may perform one or more of addition, subtraction, multiplication and division on the normalized scores. In some aspects, transformation engine 406 inputs scores 408 into a predefined function that weights the each of the scores 408. The output of transformation engine 406 is a final score 410.

In some aspects, each scoring model 404-1 to 404-N may further output an uncertainty 412-1 to 412-N (N being a positive whole integer greater than 1; collectively uncertainties 412). Uncertainties 412 may be due to differences between values calculated by scoring models 404 and experimental values. Uncertainties 412 may be determined using statistical methods (e.g., root mean square error and the like). Transformation engine 406 may perform operations, such as error propagation, on uncertainties 412 to generate a single uncertainty value for final score 410. The uncertainty may be added or subtracted from final score 410. In some aspects, when scoring models 404 do not output uncertainty, a threshold value (e.g., as described in 206 of process 200 in FIG. 2) may act as a proxy for uncertainty.

In the examples above, scoring models and transformations for a materials generation process have been described. However, these examples are not meant to be limiting nor meant to represent an exhaustive list of possible implementations. Specifically, the concepts described herein may be applied to other engineering design problems where a lower fidelity and approximate scoring model may be defined. For example, if materials generation platform 102 were applied to the design of an airplane wing, a scoring model may measure friction on the airplane wing. The scope of the technology disclosed herein is not limited to only these examples, and other implementations are contemplated as appreciated by one skilled in the art.

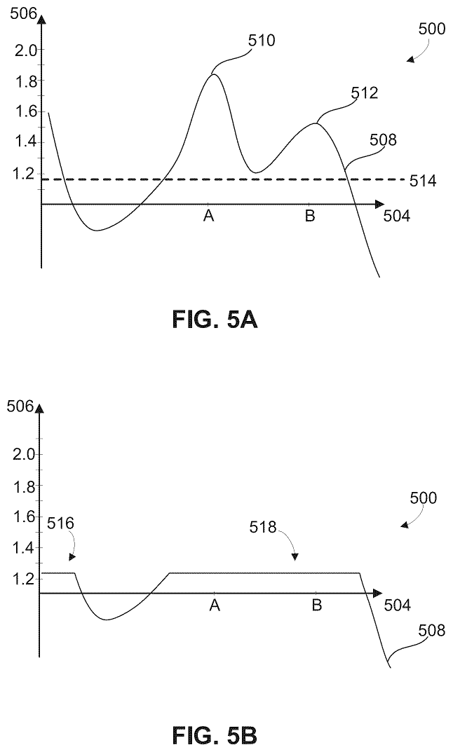

FIGS. 5A and 5B show an example plot 500, according to some aspects. Plot 500 may display scores for multiple candidate materials. Scores may be calculated as described above in reference to FIG. 4. In plot 500, candidate materials are depicted on axis 504 (i.e., each “point” on axis 502 is a candidate material), scores are depicted on axis 506 in arbitrary units and data 508 plots the scores for each candidate material. Data 508 exhibits a global maximum value at 510, corresponding to material A, and a local maximum value at 512, corresponding to material B.

Plot 500 may further include a maximum threshold value 514. Threshold value 514 may be determined from the uncertainty (e.g., degree of belief) of the scoring models used to generate data 508. For example, threshold value 514 may be determined from statistical error, such as root mean square error (RMSE) or the like. In some aspects, threshold value 514 may indicate a value above which scores are considered identical due to uncertainty present in the scoring models. Additionally or alternatively, the threshold value may define a minimum difference between scores that is needed for the scores to be considered distinct. In some aspects, the difference between two scores is compared to a threshold value. If the difference is less than the threshold value, the two scores are considered equal.

FIG. 5B shows plot 500 after scores above threshold value 514 are truncated. Truncating results in identical scores in regions 516 and 518. The global and local maxima at 510 and 512 are no longer present. In some aspects, applying the threshold value 514 to data 508 reduces the risk of artificial maxima and minima and allows for exploration of a larger material space. For example, material A has a higher score than material B before the threshold is applied. When threshold value 514 is not applied, material A will always be chosen a best candidate material over material B in step 208 of process 200. However, after applying threshold value 514, materials A and B have an equal scores and an equal probability of being chosen as a best candidate material (see FIG. 2, step 208). As shown herein, accounting for uncertainty allows a materials generation platform to consider a larger pool of candidate materials, which gives a higher likelihood of generating novel material structures.

While, plot 500 in FIGS. 5A and 5B illustrate a continuum of scores for a plurality of candidate materials, it will be understood by a person of ordinary skill in the art that the concepts described herein may be applied to as few as two candidate materials.

In the examples above, applying uncertainty to scores generated a materials generation process have been described. However, these examples are not meant to be limiting nor meant to represent an exhaustive list of possible implementations. The scope of the technology disclosed herein is not limited to only these examples, and other implementations are contemplated as appreciated by one skilled in the art.

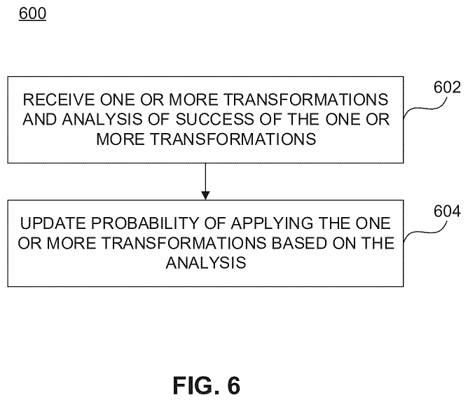

FIG. 6 shows an example process 600, according to some aspects. Process 600 may describe methods for implementing active learning in a materials generation platform. Operations described may be implemented by processing logic that may comprise hardware (e.g., circuitry, dedicated logic, programmable logic, microcode, etc.), software (e.g., instructions executing on a processing device), firmware or a combination thereof. It is to be appreciated that not all operations may be needed to perform the disclosure provided herein. Further, some of the operations may be performed simultaneously, or in a different order than described for FIG. 6, as will be understood by a person of ordinary skill in the art. Process 600 shall be described with reference to FIGS. 1-5. However, process 600 is not limited to these example aspects.

At 602, an active learning engine (e.g., active learning engine 112) of a materials generation platform (e.g., materials generation platform 102) may receive a set of one or more transformations used to generate a second candidate material from a first candidate material. The active learning engine may also receive analysis (e.g., from analysis engine 110) on whether the first candidate material or the second candidate material is the best candidate material (see step 208 of FIG. 2). Alternatively, the second candidate material may be compared to a threshold (e.g., a stability threshold), and the active learning engine may receive analysis on whether a score of the second candidate material is above or below the threshold.

At 604, the active learning engine may update a probability of applying the one or more transformations based on the analysis. For example, if the second candidate material is the best candidate material, the one or more transformations may have resulted in a material structure that better meets the objectives scored by a set of scoring models (see FIG. 4). Because the one or more transformations resulted in a better material, the probability of applying the one or more transformations may increase. Alternatively, if the first candidate material is the best candidate material, the one or more transformation resulted in a worse material structure and the probability of applying the one or more transformations may decrease.

When a score of the second candidate material is compared to a threshold (instead of a score of the first candidate material) the active learning engine may update the probability of applying the one or more transformations based on whether the score of the second candidate material falls above or below the threshold. For example, if a value below the threshold is favorable, the active learning engine may increase the probability of applying the one or more transformations when the score is below the threshold and decrease the probability of applying the one or more transformations when the score is above the threshold.

In some aspects, 602 and 604 are repeated after each iteration of a materials generation simulation, thus allowing the materials generation platform to “learn” which transformations are more likely to give favorable results (e.g., new materials with the desired properties). Furthermore, as the probability of applying a transformation increases, the transformation is more likely to be applied to a candidate material. This allows the materials generation platform to generate novel materials more efficiently.

FIG. 7 depicts an example computer system 700 useful for implementing various aspects described herein.

Various aspects may be implemented, for example, using one or more well-known computer systems, such as computer system 700 shown in FIG. 7. One or more computer systems 700 may be used, for example, to implement any of the aspects discussed herein, as well as combinations and sub-combinations thereof. Cloud implementations may include one or more of the example computer systems operating locally or distributed across one or more server sites.

Computer system 700 may include one or more processors (also called central processing units, or CPUs), such as a processor 704. Processor 704 may be connected to a communication infrastructure or bus 706.

Computer system 700 may also include customer input/output device(s) 702, such as monitors, keyboards, pointing devices, etc., which may communicate with communication infrastructure 706 through customer input/output interface(s) 702.

One or more of processors 704 may be a graphics processing unit (GPU). In an aspect, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 may also include a main or primary memory 708, such as random access memory (RAM). Main memory 708 may include one or more levels of cache. Main memory 708 may have stored therein control logic (i.e., computer software) and/or data.

Computer system 700 may also include one or more secondary storage devices or memory 710. Secondary memory 710 may include, for example, a hard disk drive 712 and/or a removable storage device or drive 714. Removable storage drive 714 may be a floppy disk drive, a magnetic tape drive, a compact disk drive, an optical storage device, tape backup device, and/or any other storage device/drive.

Removable storage drive 714 may interact with a removable storage unit 716. Removable storage unit 716 may include a computer usable or readable storage device having stored thereon computer software (control logic) and/or data. Removable storage unit 716 may be a floppy disk, magnetic tape, compact disk, DVD, optical storage disk, and/any other computer data storage device. Removable storage drive 714 may read from and/or write to removable storage unit 716.

Secondary memory 710 may include other means, devices, components, instrumentalities or other approaches for allowing computer programs and/or other instructions and/or data to be accessed by computer system 700. Such means, devices, components, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 700 may further include a communication or network interface 724. Communication interface 724 may enable computer system 700 to communicate and interact with any combination of external devices, external networks, external entities, etc. (individually and collectively referenced by reference number 728). For example, communication interface 724 may allow computer system 700 to communicate with external or remote devices 728 over communications path 726, which may be wired and/or wireless (or a combination thereof), and which may include any combination of LANs, WANs, the Internet, etc. Control logic and/or data may be transmitted to and from computer system 700 via communication path 726.

Computer system 700 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 700 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 700 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML Customer Interface Language (XUL), or any other functionally similar representations alone or in combination. Alternatively, proprietary data structures, formats or schemas may be used, either exclusively or in combination with known or open standards. In some aspects, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 700, main memory 708, secondary memory 710, and removable storage units 716 and 722, as well as tangible articles of manufacture embodying any combination of the foregoing. Such control logic, when executed by one or more data processing devices (such as computer system 700), may cause such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 7. In particular, aspects can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A computer implemented method, comprising:
generating, by applying one or more transformations to a first material candidate, a second material candidate, wherein the one or more transformations are restricted by a set of constraints;
scoring, using a scoring function, the first material candidate and the second material candidate;
truncating results of the scoring function based on a threshold value, wherein the threshold value is related to uncertainty within the scoring function;
choosing a best material candidate from the first material candidate or the second material candidate based on a score for each material candidate after the truncating; and
designing a material structure comprising at least the best material candidate.

2. The computer implemented method of claim 1, further comprising:
generating, by applying another set of one or more transformations to the best material candidate, a third material candidate;
scoring, using the scoring function, the best material candidate and the third material candidate;
truncating results of the scoring function based on the threshold value; and
choosing another best material candidate from the best material candidate or the third material candidate based on the truncating; and
using the another best material candidate in the designing.

3. The computer implemented method of claim 1, wherein the generating, scoring, truncating, and choosing are implemented for multiple pairs of candidates in parallel.

4. The computer implemented method of claim 1, wherein the first material candidate and the second material candidate are materials with unit cells comprising at least 30 atoms.

5. The computer implemented method of claim 1, wherein the first material candidate and the second material candidate are materials with unit cells comprising at least 100 atoms.

6. The computer implemented method of claim 1, wherein the scoring function is configured to score the first material candidate and the second material candidate based on desired properties of the material structure.

7. The computer implemented method of claim 1, further comprising:
increasing a probability of applying the one or more transformations in a system memory in response to the second material candidate being the best material candidate or decreasing the probability of applying the one or more transformations in a system memory in response to the first material candidate being the best material candidate; and
applying the one or more transformations to a subsequent material candidate.

8. The computer implemented method of claim 1, wherein the material structure is a symmetric oxide, a perovskite, spinel, pyrochlore, or a garnet.

9. A system, comprising:
a memory; and
a processor configured to:
generate, by applying one or more transformations to a first material candidate, a second material candidate, wherein the one or more transformations are restricted by a set of constraints;
score, using a scoring function, the first material candidate and the second material candidate;
truncate results of the scoring function based on a threshold value, wherein the threshold value is related to uncertainty within the scoring function;
choose a best material candidate from the first material candidate or the second material candidate based on the truncating; and
design a material structure comprising at least the best material candidate.

10. The system of claim 9, wherein the processor is further configured to:
generate, by applying another set of one or more transformations to the best material candidate, a third material candidate;
score, using the scoring function, the best material candidate and the third material candidate;
truncate results of the scoring function based on the threshold value; and
choose another best candidate material from the best material candidate or the third material candidate based on the truncating.

11. The system of claim 9, wherein the processor is further configured to:
implement the generating, scoring, truncating, and choosing for multiple pairs of material candidates in parallel.

12. The system of claim 9, wherein the first material candidate and the second material candidate are materials with unit cells comprising at least 30 atoms.

13. The system of claim 9, wherein the first material candidate and the second material candidate are materials with unit cells comprising at least 100 atoms.

14. The system of claim 9, wherein the scoring function is configured to score the first material candidate and the second material candidate based on desired properties.

15. The system of claim 9, wherein the processor is further configured to:
increase a probability of applying the one or more transformations in a system memory in response to the second material candidate being the best material candidate or decrease a probability of applying the one or more transformations in a system memory in response to the first material candidate being the best material candidate; and
apply the one or more transformations to a subsequent material candidate.

16. The system of claim 9, wherein the material structure is a symmetric oxide, a perovskite, spinel, pyrochlore, or a garnet.

17. A non-transitory machine readable storage medium having instructions stored thereon that, when executed by a set of one or more processors, cause said set of one or more processors to perform operations comprising:
generating, by applying one or more transformations to a first material candidate, a second material candidate, wherein the one or more transformations are restricted by a set of constraints;
scoring, using a scoring function, the first material candidate and the second material candidate;
truncating results of the scoring function based on a threshold value, wherein the threshold value is related to uncertainty within the scoring function;
choosing a best material candidate from the first material candidate or the second material candidate based on a score for each material candidate after the truncating; and
designing a material structure comprising at least the best material candidate.

18. The non-transitory machine readable storage medium of claim 17, wherein the operations further comprise:
generating, by applying another set of one or more transformations to the best material candidate, a third material candidate;
scoring, using the scoring function, the best material candidate and the third material candidate;
truncating results of the scoring function based on the threshold value;
choosing another best material candidate from the best material candidate or the third material candidate based on the truncating; and
using the another best material candidate in the designing.

19. The non-transitory machine readable storage medium of claim 17, wherein the operations further comprise:
implementing the generating, scoring, truncating, and choosing for multiple pairs of material candidates in parallel.

20. The non-transitory machine readable storage medium of claim 17, wherein the operations further comprise:
increasing a probability of applying the one or more transformations in a system memory in response to the second material candidate being the best material candidate or decreasing the probability of applying the one or more transformations in a system memory in response to the first material candidate being the best material candidate; and
applying the one or more transformations to a subsequent material candidate.
